(19) United States
(12) Patent Application Publication
(10) Pub. No.: US 2013/0116580 A1
(43) Pub. Date: May 9, 2013

(54) SYSTEM FOR QUALITY ASSESSMENT OF PHYSIOLOGICAL SIGNALS AND METHOD THEREOF

(71) Applicants: Jia Liu, Guangdong (CN); Pandeng Zhang, Guangdong (CN); Xiaochang Liu, Guangdong (CN); Xiaoan Gu, Guangdong (CN); Jingyu Yan, Guangdong (CN); Xinyu Wu, Guangdong (CN); Yangsheng Xu, Guangdong (CN)

(72) Inventors: Jia Liu, Guangdong (CN); Pandeng Zhang, Guangdong (CN); Xiaochang Liu, Guangdong (CN); Xiaoan Gu, Guangdong (CN); Jingyu Yan, Guangdong (CN); Xinyu Wu, Guangdong (CN); Yangsheng Xu, Guangdong (CN)

(73) Assignee: Shenzhen Institute of Advanced Technology Chinese Academy of Sciences, GUANGDONG (CN)

(21) Appl. No.: 13/701,537

(22) PCT Filed: Jul. 2, 2010
(86) PCT No.: PCT/CN10/74925

§ 371 Date: Jan. 15, 2013

(30) Foreign Application Priority Data
Jun. 4, 2010 (CN) CN201010192921.4

(51) Int. Cl. A61B 5/04; A61B 5/0402; A61B 5/021; A61B 5/024; A61B 5/00; A61B 5/0215

(52) U.S. Cl. CPC A61B 5/04017; A61B 5/7225; A61B 5/7267; A61B 5/0215; A61B 5/021; A61B 5/024; A61B 5/7203; A61B 5/0402
USPC 600/486; 600/300; 600/485; 600/500

(57) ABSTRACT

The present disclosure relates to a system for physiological signal quality assessment, the system includes: a first filter module for implementing a filter process on an inputted first physiological signal; a first periodicity detection module for detecting periodicity of the filtered first physiological signal, and determining periodic segmentation point of the first physiological signal; a feature extracting module for extracting corresponding signal features of the first physiological signal in each heart period; and a fuzzy logic module for building up a fuzzy logic model according to the extracted signal features, and calculating a signal quality index for the first physiological signal in the relative period based on the built fuzzy logic model, and determining a signal attribute according to the signal quality index. A method for physiological signal quality assessment is provided as well. The system and method for physiological signal quality assessment calculate the signal quality index, determine the signal attribute according to the signal quality index, therefore recognize the abnormal signal out of the first physiological signal, and result in high quality physiological signals.

### FIELD OF THE INVENTION

The present disclosure relates generally to the field of computer-based medical application technology, and more particularly, to a system for quality assessment of physiological signals and a method thereof.

### BACKGROUND OF THE INVENTION

Arterial Blood Pressure (ABP) signal is a common physiological signal, the continuous measurement and analysis of which is of high significance to the clinical diagnostic of hypertension and analysis of the automatic adjustment function of cerebral blood flow. The continuous measurement includes two kinds: the invasive one and the noninvasive one. The invasive continuous ABP measurement has high liability and stability, but it needs to be embedded into the body and requires aseptic conditions; therefore the usage is limited in particular situations, such as a surgery room. Comparatively, the noninvasive continuous ABP measurement has many advantages such as measurement convenience, operation simplicity, noninvasive and no requirement for aseptic; therefore the noninvasive continuous ABP measurement method is getting more and more widely used.

There are a few methods for the noninvasive continuous ABP measurement; the tension-determination method and the volume-compensation method are two developed kinds of noninvasive continuous ABP measurement method. The measurement position of the method is on the limb ends (fingertips or radial arteries), thus the measurements tend to be affected by the external, with instability of the signals increasing; therefore, the ABP signals shall be carefully used, and it is quite necessary to build up an assessment method for the clinical ABP signal quality.

Currently, in the noninvasive continuous ABP measurement, two kinds of pseudo-difference signals need to be solved: 1) abnormality signal calibration caused by the pressure calibration of the measurement instrument; 2) motion abnormal signal or signal absence produced by the shift or jitter of the sensor due to the patient's posture change or movement. The pseudo-difference signals are generated by the abnormal signals caused by the instruments (such as poor contact) instead of the physiological changes of the patient. The pseudo-difference signals have rather large volatility and lack useful information, sequentially causing high volatility and poor repeatability of the follow-up analysis results which moreover could not be fundamentally recovered through ordinary wave-filtering and estimation methods.

### SUMMARY OF THE INVENTION

Accordingly, it is necessary to provide a system for quality assessment of physiological signals, to obtain high quality physiological signals.

Besides, it is necessary to provide a method for quality assessment of physiological signals, to obtain high quality physiological signals.

A system for quality assessment of physiological signals includes:

a first filter module for implementing a wave-filter process on an inputted first physiological signal;

a first periodicity detection module for detecting periodicity of the wave-filtered first physiological signal, and determining periodic segmentation point of the first physiological signal;

a feature extraction module for extracting corresponding signal features of the first physiological signal in each heart period; and

a fuzzy logic module for building up a fuzzy logic model according to the extracted signal features, and calculating a signal quality index for the first physiological signal in the relative period based on the built fuzzy logic model, and determining a signal attribute according to the signal quality index.

Preferably, the first physiological signal is invasive continuous arterial blood pressure signal, noninvasive continuous arterial blood pressure signal, or pulse signal.

Preferably, the filter process on the first physiological signal is to filter noise with frequency higher than 40 Hz out from the first physiological signal.

Preferably, the feature extraction module further sets up a membership function for the extracted signal features, the membership function is:

$$S ( x ; a , b ) = \{ \begin{matrix}0 , & x \leq a \\ 2 {( \frac{x - a}{b - a} )}^{2} , & a < x \leq \frac{a + b}{2} \\ 1 - 2 {( \frac{x - b}{b - 1} )}^{2} , & \frac{a + b}{2} < x \leq b \\ 1 , & b < x\end{matrix} ,$$

wherein x is the current feature value; a and b are parameters determined by experiment.

Preferably, the signal features include calibration abnormality signal feature u₁ and motion abnormality signal feature u₂; x in the membership of the calibration abnormality signal feature u₁ is an end-diastolic slope sum; x in the membership of the motion abnormality signal feature u₂ is a ratio of an absolute value of the difference between two successive diastolic pressures and the less value thereof.

Preferably, the system for quality assessment of physiological signal further includes:

a second filter module for implementing a wave-filter process on an inputted second physiological signal which is synchronously sampled with said first physiological signal;

a second periodicity detection module for detecting periodicity of the wave-filtered second physiological signal, and determining periodical segment points of the second physiological signal;

wherein the feature extraction module is further used for extracting signal features of the second physiological signal in the same period related to the first physiological signal.

Preferably, the second physiological signal is electrocardiogram signal.

Preferably, the filter process implemented on the second physiological signal is for filtering noise with frequency lower than 0.05 Hz or higher than 100 Hz, and 50 Hz power frequency noise.

Preferably, the extracted signal feature in relation is period normality signal feature u₃; and x in the membership of the period normality signal feature u₃ stands for a ratio of a delay time from a comprehensive peak value point of the current period electrocardiogram signal to a starting u point of the arterial blood pressure signal and a base value of the delay time.

Preferably, the fuzzy logic model built up by the fuzzy logic module according to the extracted signal features and signal features in relation is: SQI=u_{SQG}=1−u₁ [symbol] u₂ [symbol] u₃, wherein SQI is the signal quality index, [symbol] means taking a maximum value.

Preferably, the signal attribute is normal signal, abnormal signal or transition signal; the fuzzy logic module is further used for setting up a threshold value and comparing the signal quality index with said threshold; the first physiological signal of the relative period is a normal signal if the signal quality index is larger than the threshold value, the first physiological signal of the relative period is a transition signal if the signal quality index equals the threshold value, the first physiological signal of the relative period is an abnormal signal if the signal quality index is lower than the threshold value.

A method for quality assessment of physiological signals includes:

implementing a wave-filter process on an inputted first physiological signal;

implementing a periodicity detection on the wave-filtered first physiological signal, and determining periodic segmentation points of the first physiological signal;

extracting corresponding signal features from the first physiological signal in every period circles;

building up a fuzzy logic model according to the extracted signal features; calculating a signal quality index for the first physiological signal in the relative period based on the built fuzzy logic model; and determine a signal attribute according to the signal quality index.

Preferably, the first physiological signal is invasive continuous arterial blood pressure signal, noninvasive continuous arterial blood pressure signal, or pulse signal.

Preferably, the filter process on the first physiological signal is to filter noise with frequency higher than 40 Hz out from the first physiological signal.

Preferably, the method further includes: setting up a membership function for the extracted signal features, the membership function is:

$$S ( x ; a , b ) = \{ \begin{matrix}0 , & x \leq a \\ 2 {( \frac{x - a}{b - a} )}^{2} , & a < x \leq \frac{a + b}{2} \\ 1 - 2 {( \frac{x - b}{b - 1} )}^{2} , & \frac{a + b}{2} < x \leq b \\ 1 , & b < x\end{matrix} ,$$

wherein x is the current feature value; a and b are parameters determined by experiment.

Preferably, the signal features include calibration abnormality signal feature u₁ and motion abnormality signal feature u₂; x in the membership of the calibration abnormality signal feature u₁ is an end-diastolic slope sum; x in the membership of the motion abnormality signal feature u₂ is a ratio of an absolute value of the difference between two successive diastolic pressures and the less value thereof.

Preferably, the method further includes:

implementing a wave-filter process on an inputted second physiological signal which is synchronously sampled with the first physiological signal;

detecting periodicity of the filtered second physiological signal, and determining periodical segment points of the second physiological signal;

extracting signal features of the second physiological signal in the same period in relation to the first physiological signal.

Preferably, the second physiological signal is electrocardiogram signal.

Preferably, the wave-filter process implemented on the second physiological signal is for filtering noise with frequency lower than 0.05 Hz or higher than 100 Hz, and 50 Hz power frequency noise.

Preferably, the extracted signal feature in relation is period normality signal feature u₃; and x in the membership of the period normality signal feature u₃ stands for a ratio of a delay time from a comprehensive peak value point of the current period electrocardiogram signal to a starting u point of the arterial blood pressure signal and a base value of the delay time.

Preferably, the fuzzy logic model which is built up according to the extracted signal features and signal features in relation is: SQI=u_{SQG}=1−u₁ [symbol] u₂ [symbol] u₃, wherein SQI is the signal quality index, [symbol] means taking a maximum value.

Preferably, the signal attribute is normal signal, abnormal signal or transition signal; the method further includes: setting up a threshold value and comparing the signal quality index with the threshold value; the first physiological signal of the relative period is a normal signal if the signal quality index is larger than the threshold value, the first physiological signal of the relative period is a transition signal if the signal quality index equals the threshold value, the first physiological signal of the relative period is an abnormal signal if the signal quality index is lower than the threshold value.

The above described system for physiological signal quality assessment and method thereof carry on a filter process on the inputted first physiological signal and determine the period segment points, extract the related signal features in every signal period, and calculate the signal quality index according to the signal features, and further determine the signal attributes according to the signal quality index, and therefore recognize the abnormal signal out of the first physiological signal, and result in high quality physiological signals.

Besides, the second physiological signal is used for reference, which improves the accuracy of the signal quality index calculation; therefore the recognition rate of the abnormal signal is improved, and further even better physiological signal quality is obtained.

### BRIEF DESCRIPTION OF THE DRAWINGS

FIG. 1 is a schematic structural diagram showing a system for quality assessment of physiological signals of one embodiment.

FIG. 2 is a signal chart of normal and abnormal noninvasive continuous ABP signals measured through tension-determination method.

FIG. 3 is a schematic diagram of the EDSS feature principle.

FIG. 4 is a schematic structural diagram showing a system for quality assessment of physiological signals of another embodiment.

FIG. 5 is a flow chart of a method for quality assessment of physiological signals of one embodiment.

FIG. 6 is a flow chart of a method for quality assessment of physiological signals of another embodiment.

FIG. 7 is an effect diagram of fuzzy recognition.

### DESCRIPTION OF THE PREFERRED EMBODIMENTS

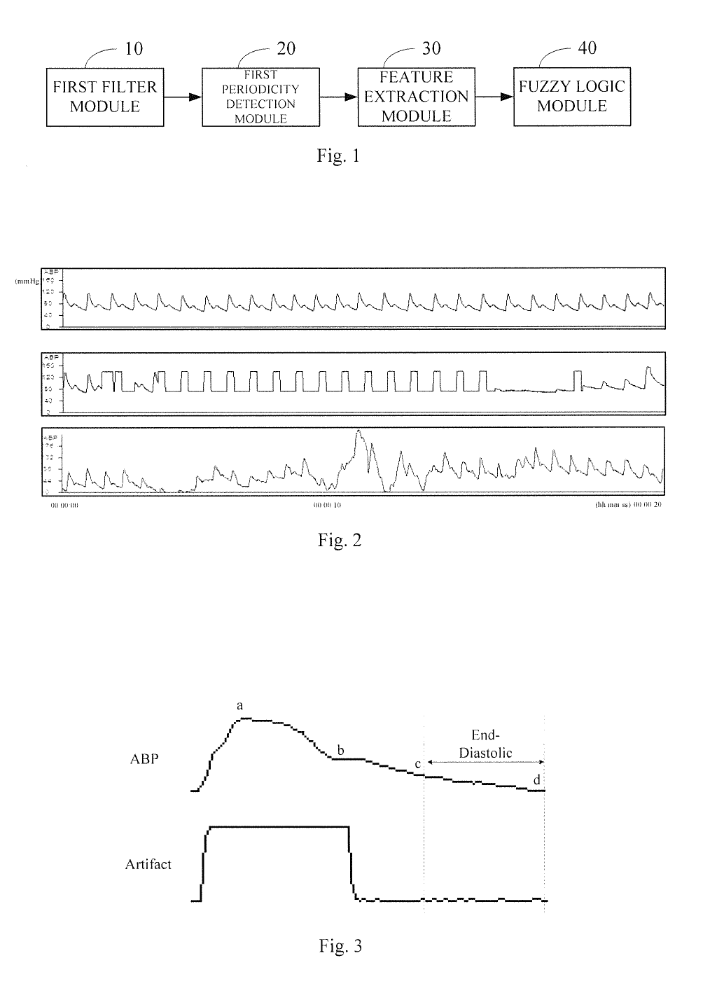

Referring to FIG. 1, a system for quality assessment of physiological signals includes a first filter module 10, a first periodicity detection module 20, a feature extraction module 30, and a fuzzy logic module 40. Wherein,

The first filter module 10 wave-filters an inputted first physiological signal. In the present embodiment, the first filter module 10 is an ABP (Arterial Blood Pressure) low-pass filter; the first physiological signal is the ABP signal which is a kind of noninvasive continuous ABP signal measured through tension-determination method. An ABP signal typically includes a pseudo-difference signal and a normal signal, both of which generally have the same signal features as periodicity, shrink and expand, with reference to FIG. 2. Wherein, the pseudo-difference signal is mixed by noise and a normal signal. The ABP low-pass filter filters out noise with frequency higher than 40 Hz in the ABP pseudo-difference signals. Besides, the first physiological signal could be invasive continuous ABP signal, pulse signal, or other physiological signals as well.

The first periodicity detection module 20 detects periodicity of the post-filtered first physiological signal, and determines periodic segmentation point of the first physiological signal. In the present embodiment, the first periodicity detection module 20 is an ABP periodicity detector. After the ABP low-pass filter filters out the noise with higher than 40 Hz frequency in the ABP pseudo-difference signals, the ABP periodicity detector is used for detecting the periodic segmentation points of the ABP pseudo-difference signals, and used for segmenting the ABP pseudo-difference signals into periodical signals one by one.

The feature extraction module 30 extracts relative signal features of the first physiological signal in each heart period. The feature extraction module 30 is an ABP feature extractor. The ABP feature extractor extracts relative signal features from the ABP pseudo-difference signals that have been divided into periodical signals; the signal features include calibration abnormality signal feature u₁, motion abnormality signal feature u₂. In a preferred embodiment, the feature extraction module 30 also sets up a membership function for the extracted signal features. The membership function is:

$$S ( x ; a , b ) = \{ \begin{matrix}0 , & x \leq a \\ 2 {( \frac{x - a}{b - a} )}^{2} , & a < x \leq \frac{a + b}{2} \\ 1 - 2 {( \frac{x - b}{b - 1} )}^{2} , & \frac{a + b}{2} < x \leq b \\ 1 , & b < x\end{matrix} ,$$

Wherein, x is the current feature value; a and b are parameters determined by experiment.

Calculate the signal feature value of the current period for the first physiological signal as followings:

x in the membership of the calibration abnormality signal feature u₁ is the End-Diastolic Slope Sum (EDSS), and the calculation equation is

$$E D S S = {\sum }_{i : c \to d}^{ } Δ {y}_{i} ,$$

wherein Δyᵢ=yᵢ−yᵢ₋₁, yᵢ is the value of the ABP pseudo-difference signal at i time point (sampling point). FIG. 3 illustrates the EDSS feature principle.

x in the membership of the motion abnormality signal feature u₂ is the ratio of an absolute value of a difference between two successive diastolic pressures and a less value thereof, i.e. x is |ΔDBP|/min(DBPᵢ, DBPᵢ₋₁).

The fuzzy logic module 40 builds up a fuzzy logic model according to the extracted signal features; and calculates a signal quality index for the first physiological signal in the relative period based on the built fuzzy logic model; and determines a signal attribute according to the signal quality index. On the basis of the extracted signal features, which are the calibration abnormality signal feature u₁, and the motion abnormality signal feature u₂, the fuzzy logic module 40 sets up semantic variables and fuzzy semantic rules; and sequentially builds up the fuzzy logic model to carry out the quality assessment of the ABP pseudo-difference signal, which means calculating the signal quality index (SQI) for the ABP pseudo-difference signal in the corresponding period.

Structure of the built fuzzy logic model is SQI=u_{SQG}=1−u₁ [symbol] u₂, wherein SQI is the signal quality index, the larger one between u₁and u₂ is incorporated. Accordingly, through processing of the ABP pseudo-difference signal, the recognition rate of normal signals out of the abnormal signals could be higher than 90%.

In the present embodiment, the signal attributes are normal signal, abnormal signal, or transition signal. The fuzzy logic module 40 sets up a threshold value, and compares the signal quality index with the threshold value. In case that the signal quality index is higher than the threshold value, the ABP pseudo-difference signal in the current period is a normal signal; if the signal quality index equals the threshold value, the ABP pseudo-difference signal in the current period is a transition signal; and provided the signal quality index is lower than the threshold value, the ABP pseudo-difference signal in the current period is an abnormal signal.

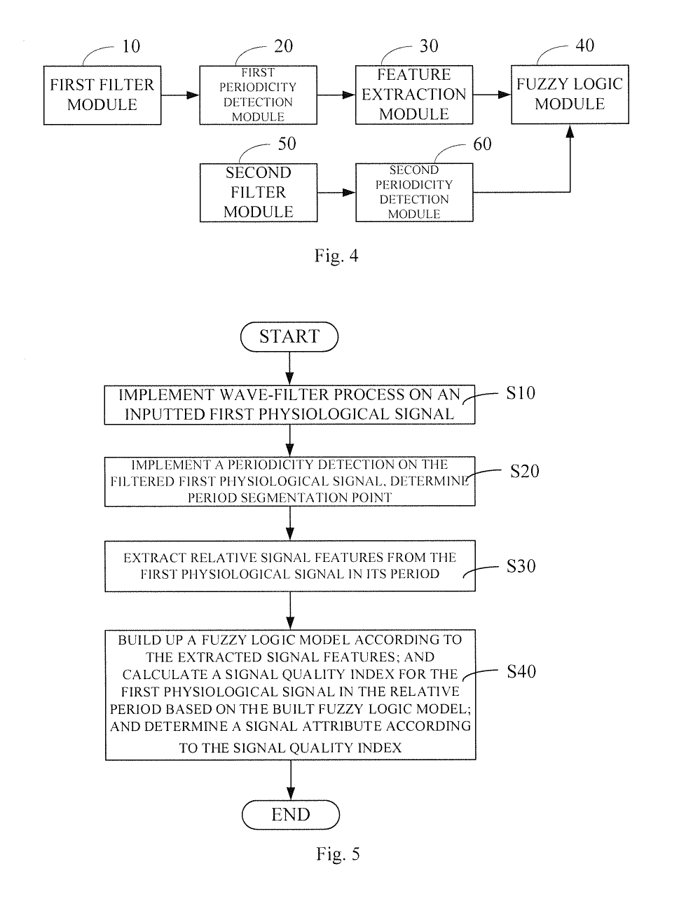

According to one embodiment, referring to FIG. 4, the system for quality assessment of physiological signals further includes a second filter module 50 and a second periodicity detection module 60. The second filter module 50 carries out a filter process on an inputted second physiological signal which is synchronously sampled with the first physiological signal. In the present embodiment, the second filter module 50 is an electrocardiogram (ECG) filter. The second physiological signal is an ECG signal. The ECG signal is synchronously sampled together with the ABP pseudo-difference signal, and is a reference signal for the ABP pseudo-difference signal. The ECG filter filters noise with frequency lower than 0.05 Hz or higher than 100 Hz out from the ECG signal, as well as the 50 Hz power frequency noise. The second periodicity detection module 60 detects periodicity of the post-filtered second physiological signal, and determines the periodical segment point of the second physiological signal. That is, the second periodicity detection module 60 detects periodicity of the post-filtered ECG signal, and segments the ECG signals into periodical signals one by one.

According the present embodiment, the signal feature of the second physiological signal relative to the first physiological signal in the same period as well as the calibration abnormality signal feature u₁ and the motion abnormality signal feature u₂ are extracted by the feature extraction module 30. The signal feature in relation is the period normality signal feature u₃. In the membership of the period normality signal feature u₃, x stands for the ratio of a delay time from a comprehensive peak value point of the current period ECG signal to a starting u point of the ABP signal and a base value of the delay time, wherein DTa is the base value of DT, and DTa=w₁×DTi+w₂×DTa, w₁ and w₂ are constants.

According to the present embodiment, the number of the samples is 78, the membership functions of the signal features are respectively:

u₁=S(EDSS;−12,0);

u₂=S(|ΔDBP|/min(DBPᵢ,DBPᵢ₋₁);1,3);

u₃=S(DT/DTa;0.4,0.9) [symbol] (1−S(DT/DTa;1.1,1.6)); wherein [symbol] means taking the least value.

And wherein, DTa=w₁×DTi+w₂×DTa, w₁ and w₂ are constants, w₁ is 0.125, and w₂ is 0.875.

The fuzzy logic module 40 sets up semantic variables and fuzzy semantic rules according to the extracted signal features and the relative signal features; and further builds up a fuzzy logic model as SQI=u_{SQG}=1−u₁ [symbol] u₂ [symbol] u₃, wherein SQI is the signal quality index, the largest among u₁, u₂ and u₃ is incorporated. The SQI is calculated through introducing the calculated calibration abnormality signal feature u₁, motion abnormality signal feature u₂, and period normality signal feature u₃ into the model. Whereas, the fuzzy semantic rule is recorded with a form as referred in table 1.

TABLE 1
Fuzzy Semantic Rule Table

| Feature u₁ | Feature u₂ | Feature u₃ | Type of Signal |
|---|---|---|---|
| Low | Low | Normal | Normal Signal |
| Large | — | — | Calibration Abnormal and |
| | | | Loss Signal |
| — | Large | — | Motion Abnormal Signal |

Referring to FIG. 5, according to one embodiment, a method for quality assessment of physiological signals includes the following steps:

Step S10, implementing a wave-filter process on an inputted first physiological signal. In the present embodiment, the first filter module is used for implementing the filter process on the first physiological signal. Wherein, the first filter module is the ABP (Arterial Blood Pressure) low-pass filter; the first physiological signal is the ABP signal which is a kind of noninvasive continuous ABP signal measured through tension-determination method. The ABP signal includes a pseudo-difference signal and a normal signal, both of which have the same signal features as periodicity, shrink and expand. Wherein, the pseudo-difference signal is mixed by noise and a normal signal. The ABP low-pass filter filters out noise with frequency higher than 40 Hz from the ABP pseudo-difference signal. Besides, the first physiological signal could be an invasive continuous ABP signal, pulse signal, or other physiological signals as well.

Step S20, implementing a periodicity detection on the post-filtered first physiological signals. After the ABP low-pass filter filters out the noise with over 40 Hz frequency in the ABP pseudo-difference signal, the ABP periodicity detector is used for detecting the periodic segmentation point of the ABP pseudo-difference signal, and for segmenting the ABP pseudo-difference signal into periodical signals one by one.

Step S30, extracting corresponding signal features from the first physiological signal in each heart period (cycle). In the present embodiment, an ABP feature extractor is used for extracting corresponding signal features from the ABP pseudo-difference signal that have been divided into periodical signals; the signal features include calibration abnormality signal feature u₁, motion abnormality signal feature u₂. In a preferred embodiment, the method further includes a step of setting up membership functions for the extracted signal features. The membership function is:

$$S ( x ; a , b ) = \{ \begin{matrix}0 , & x \leq a \\ 2 {( \frac{x - a}{b - a} )}^{2} , & a < x \leq \frac{a + b}{2} \\ 1 - 2 {( \frac{x - b}{b - 1} )}^{2} , & \frac{a + b}{2} < x \leq b \\ 1 , & b < x\end{matrix} ,$$

wherein x is, the current feature value; a and b are parameters determined by experiment.

Calculate the signal feature value of the current period of the first physiological signal, as followings:

x in the membership of the calibration abnormality signal feature u₁ is end-diastolic slope and (EDSS), the calculation equation is

$$E D S S = {\sum }_{i : c \to d}^{ } Δ {y}_{i} ,$$

wherein Δyᵢ=yᵢ−yᵢ₋₁, yᵢ is the value of the ABP pseudo-difference signal at i time point (sampling point). FIG. 3 illustrates the EDSS feature principle.

x in the membership of the motion abnormality signal feature u₂ is the ratio of an absolute value of the difference between two successive diastolic pressures and the less value thereof, i.e. x is |ΔDBP|/min(DBPᵢ,DBPᵢ₋₁).

Step S40, building up a fuzzy logic model according to the extracted relative signal features; and calculating a signal quality index for the first physiological signal in the relative period based on the built fuzzy logic model; and determining a signal attribute according to the signal quality index. On basis of the extracted signal features, which are the calibration abnormality signal feature u₁, and the motion abnormality signal feature u₂, semantic variables and fuzzy semantic rules are set up; and sequentially a fuzzy logic model is built to carry out the quality assessment of the ABP pseudo-difference signal, which means calculating the signal quality index (SQI) for the ABP pseudo-difference signal in the corresponding period.

Structure of the built fuzzy logic model is SQI=u_{SQG}=1−u₁ [symbol] u₂, wherein SQI is the signal quality index, the larger one between u₁ and u₂ is incorporated.

In the present embodiment, the signal attributes are normal signal, abnormal signal, or transition signal. The fuzzy logic module 40 sets up a threshold value, and compares the signal quality index with the threshold value. In case that the signal quality index is higher than the threshold value, the ABP pseudo-difference signal in the current period is a normal signal; if the signal quality index equals the threshold value, the ABP pseudo-difference signal in the current period is a transition signal; and provided the signal quality index is lower than the threshold value, the ABP pseudo-difference signal in the current period is an abnormal signal.

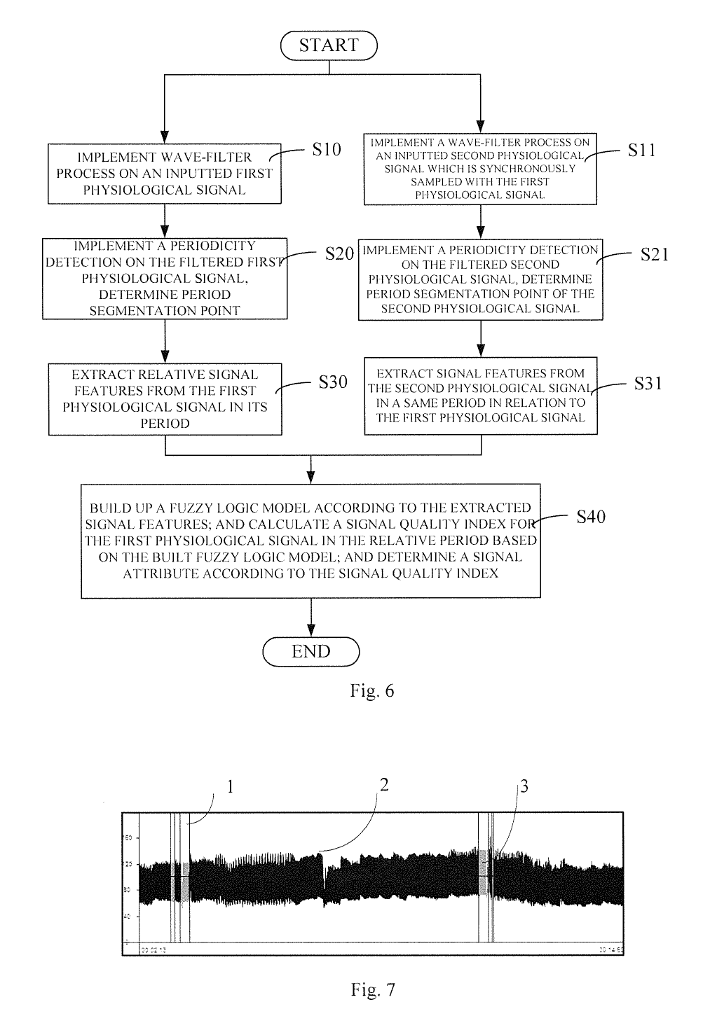

According to one embodiment, referring to FIG. 6, the method for quality assessment of physiological signals further includes the following steps:

Step S11, implementing a filter process on an inputted second physiological signal which is synchronously sampled with the first physiological signal. Wherein, the second filter module 50 is used for implementing the filter process on the second physiological signal which is synchronously sampled with the first physiological signal. In the present embodiment, the second filter module 50 is an electrocardiogram (ECG) filter. The second physiological signal is an ECG signal. The ECG signal is synchronously sampled together with the ABP pseudo-difference signal, and is a reference signal for the ABP pseudo-difference signal. The ECG filter filters noise with frequency lower than 0.05 Hz or higher than 100 Hz out from the ECG signal, as well as the 50 Hz power frequency noise.

Step S21, detecting periodicity of the post-filtered second physiological signal, and segment the periods of the second physiological signal. The second periodicity detection module 60 is used for detecting periodicity of the post-filtered second physiological signal, and determining the periodical segment point of the second physiological signal. That is, the second periodicity detection module 60 detects periodicity of the post-filtered ECG signal, and segments the ECG signals into periodical signals one by one.

Step S31, extracting signal features of the second physiological signal in relation to the first physiological signal in the same period. According to the present embodiment, the extracted signal features include the calibration abnormality signal feature u₁ and the motion abnormality signal feature u₂, as well as the period normality signal feature u₃. In the membership of the period normality signal feature u₃, x stands for the ratio of a delay time from a comprehensive peak value point of the current period ECG signal to a starting u point of the ABP signal and a base value of the delay time, wherein DTa is the base value of DT, and DTa=w₁×DTi+w₂×DTa, w₁ and w₂ are constants.

Steps S11, S21 and S31 could be executed synchronously with steps S10, S20, and S30, or could be executed after step S30.

Therefore, after extracting the signal features such as the calibration abnormality signal feature u₁ and the motion abnormality signal feature u₂, and the period normality signal feature u₃, step S40 would be transferred into step S41: building up a fuzzy logic model according to the extracted signal features; and calculating a signal quality index for the first physiological signal in the relative period based on the built fuzzy logic model; and determining a signal attribute according to the signal quality index.

According to the present embodiment, the number of the samples is 78, the membership functions of the signal features are respectively:

u₁=S(EDSS;−12,0);

u₂=S(|ΔDBP|/min(DBPᵢ,DBPᵢ₋₁);1,3);

u₃=S(DT/DTa;0.4,0.9) [symbol] (1−S(DT/DTa;1.1,1.6)); wherein, [symbol] means taking the least value.

And wherein, DTa=w₁×DTi+w₂×DTa, w₁ and w₂ are constants, w₁ is 0.125, and w₂ is 0.875.

According to the extracted signal features, semantic variables and fuzzy semantic rules are set up, and further, a fuzzy logic model is built up as: SQI=u_{SQG}=1−u₁ [symbol] u₂ [symbol] u₃, wherein SQI is the signal quality index, the largest among u₁, u₂ and u₃ is incorporated. The SQI is calculated through introducing the calculated calibration abnormality signal feature u₁, motion abnormality signal feature u₂, and period normality signal feature u₃ into the model; and further compares the SQI with a threshold value to determine the signal attributes, which is normal signal or abnormal signal. Effect of the fuzzy recognition is illustrated with reference to FIG. 7, wherein label 1 is a manually marked abnormal signal segment between the two vertical black lines, black solid line with label 2 is the normal signal recognized by the algorithm, and grey solid line with label 3 is the abnormal signal result recognized by the algorithm.

The above described system and method for quality assessment of physiological signals carries on a filter process on the inputted first physiological signal and determines its period segment point, extracts the related signal features in each signal period, and calculated the signal quality index according to the signal features, further determines the signal attributes according to the signal quality index, therefore recognizes the abnormal signal out of the first physiological signal, results in high quality physiological signals.

Besides, the second physiological signal is used for reference, which improves the accuracy of the signal quality index calculation; therefore the recognition rate of the abnormal signal is improved, further results in better physiological signal quality.

The above description of the exemplary embodiments of the invention has been presented only for the purposes of illustration and description and is not intended to be exhaustive or to limit the invention to the precise forms disclosed. Many modifications and variations are possible in light of the above teaching.

The above embodiments were described in order to explain the principles of the present disclosure and their descriptions were rather specific and detailed, they shall not be regarded as the limit to the scope of the present disclosure. It shall be mentioned that, alternative embodiments and improvements by those skilled in the art to which the present disclosure pertains without departing from its spirit and scope would be included in the desired protection of the present disclosure. Accordingly, the scope of the present disclosure is defined by the appended claims.

1. A system for quality assessment of physiological signals, wherein the system comprises:
a first filter module for implementing a filter process on an inputted first physiological signal;
a first periodicity detection module for detecting periodicity of the filtered first physiological signal, and determining periodic segmentation points of the first physiological signal;
a feature extraction module for extracting corresponding signal features of the first physiological signal in each heart period; and
a fuzzy logic module for building up a fuzzy logic model according to the extracted signal features, and calculating a signal quality index for the first physiological signal in the relative period based on the built fuzzy logic model, and determining a signal attribute according to the signal quality index.

2. The system for quality assessment of physiological signals according to claim 1, wherein the first physiological signal is an invasive continuous arterial blood pressure signal, a noninvasive continuous arterial blood pressure signal, or a pulse signal.

3. The system for quality assessment of physiological signals according to claim 2, wherein the filter process on the first physiological signal is to filter noise with frequencies higher than 40 Hz out from the first physiological signal.

4. The system for quality assessment of physiological signals according to claim 2, wherein the feature extraction module further sets up a membership function for the extracted signal features, the membership function is:
$$S ( x ; a , b ) = \{ \begin{matrix}0 , & x \leq a \\ 2 {( \frac{x - a}{b - a} )}^{2} , & a < x \leq \frac{a + b}{2} \\ 1 - 2 {( \frac{x - b}{b - 1} )}^{2} , & \frac{a + b}{2} < x \leq b \\ 1 , & b < x\end{matrix} ,$$
wherein x is the current feature value; a and b are parameters determined by experiment.

5. The system for quality assessment of physiological signals according to claim 4, wherein the signal features comprise signal feature of calibration abnormality u₁ and signal feature of motion abnormality u₂; x in the membership function of the signal feature of calibration abnormality u₁ is end-diastolic slope sum; x in the membership function of the signal feature of motion abnormality u₂ is a ratio of an absolute value of a difference between two successive diastolic pressures and a less value thereof.

6. The system for quality assessment of physiological signals according to claim 5, further comprising:
a second filter module for implementing a filter process on an inputted second physiological signal which is synchronously sampled with the first physiological signal;
a second periodicity detection module for detecting periodicity of the filtered second physiological signal, and determining periodical segment points of the second physiological signal;
wherein the feature extraction module is further used for extracting signal features of the second physiological signal in the same period in relation to the first physiological signal.

7. The system for quality assessment of physiological signals according to claim 6, wherein the second physiological signal is electrocardiogram signal.

8. The system for quality assessment of physiological signals according to claim 7, wherein the filter process implemented on the second physiological signal is for filtering noise with frequencies lower than 0.05 Hz or higher than 100 Hz, and 50 Hz power frequency noise.

9. The system for quality assessment of physiological signals according to claim 8, wherein the extracted signal feature in relation is signal feature of period normality u₃; and x in the membership of the signal feature of period normality u₃ stands for a ratio of a delay time from a comprehensive peak value point of the current period electrocardiogram signal to a starting u point of the arterial blood pressure signal and a base value of the delay time.

10. The system for quality assessment of physiological signals according to claim 9, wherein the fuzzy logic model built up by the fuzzy logic module according to the extracted signal features and signal features in relation is: SQI=u_{SQG}=1−u₁ [symbol] u₂ [symbol] u₃, wherein SQI is the signal quality index, [symbol] means taking a maximum value.

11. The system for quality assessment of physiological signals according to claim 1, wherein the signal attribute is normal signal, abnormal signal or transition signal; the fuzzy logic module is further used for setting up a threshold and comparing the signal quality index with the threshold; the first physiological signal of the relative period is the normal signal if the signal quality index is larger than the threshold, the first physiological signal of the relative period is the transition signal if the signal quality index equals the threshold, the first physiological signal of the relative period is the abnormal signal if the signal quality index is lower than the threshold.

12. A method for quality assessment of physiological signals, wherein the method comprises:
implementing a filter process on an inputted first physiological signal;
implementing a periodicity detection on the filtered first physiological signal, and determine periodic segmentation points of the first physiological signal;
extracting corresponding signal features from the first physiological signal in its period circles;
building up a fuzzy logic model according to the extracted signal features; calculate a signal quality index for the first physiological signal in the relative period based on the built fuzzy logic model; and determine a signal attribute according to the signal quality index.

13. The method for quality assessment of physiological signals according to claim 12, wherein the first physiological signal is invasive continuous arterial blood pressure signal, noninvasive continuous arterial blood pressure signal, or pulse signal.

14. The method for quality assessment of physiological signals according to claim 13, wherein the filter process on the first physiological signal is to filter noise with frequency higher than 40 Hz out from the first physiological signal.

15. The method for quality assessment of physiological signals according to claim 13, wherein the method further comprises: set up a membership function for the extracted signal features, the membership function is:
$$S ( x ; a , b ) = \{ \begin{matrix}0 , & x \leq a \\ 2 {( \frac{x - a}{b - a} )}^{2} , & a < x \leq \frac{a + b}{2} \\ 1 - 2 {( \frac{x - b}{b - 1} )}^{2} , & \frac{a + b}{2} < x \leq b \\ 1 , & b < x\end{matrix} ,$$
wherein x is the current feature value; a and b are parameters determined by experiment.

16. The method for quality assessment of physiological signals according to claim 15, wherein signal features comprise calibration abnormality signal feature u₁ and motion abnormality signal feature u₂; x in the membership of the calibration abnormality signal feature u₁ is end-diastolic slope sum; x in the membership of the motion abnormality signal feature u₂ is a ratio of an absolute value of a difference between two successive diastolic pressures and a less value thereof.

17. The method for quality assessment of physiological signals according to claim 16, wherein the method further comprises:
implementing a filter process on an inputted second physiological signal which is synchronously sampled with the first physiological signal;
detecting periodicity of the filtered second physiological signal, and determine periodical segment points of the second physiological signal;
extracting signal features of the second physiological signal in the same period in relation to the first physiological signal.

18. The method for quality assessment of physiological signals according to claim 17, wherein the second physiological signal is an electrocardiogram signal.

19. The method for quality assessment of physiological signals according to claim 18, wherein the filter process implemented on the second physiological signal is for filtering noise with frequency lower than 0.05 Hz or higher than 100 Hz, and 50 Hz power frequency noise.

20. The method for quality assessment of physiological signals according to claim 19, wherein the extracted signal feature in relation is period normality signal feature u₃; and x in the membership of the period normality signal feature u₃ stands for the delay time from a comprehensive peak value point of the current period electrocardiogram signal to a starting point of the arterial blood pressure signal.

21. The method for quality assessment of physiological signals according to claim 20, wherein the fuzzy logic model which is built up according to the extracted signal features and signal features in relation is:
SQI=u_{SQG}=1−u₁ [symbol] u₂ [symbol] u₃, wherein SQI is the signal quality index, [symbol] means taking a maximum value.

22. The method for quality assessment of physiological signals according to claim 12, wherein the signal attribute is normal signal, abnormal signal or transition signal; the method further comprises: setting up a threshold value and comparing the signal quality index with the threshold value; and the first physiological signal of the relative period is a normal signal if the signal quality index is larger than the threshold value, the first physiological signal of the relative period is a transition signal if the signal quality index equals the threshold value, the first physiological signal of the relative period is an abnormal signal if the signal quality index is lower than the threshold value.
